(19) United States
(12) Patent Application Publication
(10) Pub. No.: US 2026/0286916 A1
(43) Pub. Date: Sep. 24, 2026

(54) COMPREHENSIVE ENERGY SUPPLY SYSTEM WITH HYDROGEN PRODUCTION AND PREDICTION FUNCTION AND INTEGRATED WITH HYDROGEN ENERGY STORAGE BY RESIDUAL FLUE GAS OF GAS ENGINE

(71) Applicants: Zhejiang University, Hangzhou City (CN); Architectural design and research institute of Zhejiang University CO., LTD., Hangzhou City, Zhejiang Province (CN)

(72) Inventors: Yi YANG, Hangzhou City (CN); Shuai YU, Hangzhou City (CN); Haowei XING, Hangzhou City (CN); Shuqin CHEN, Hangzhou City (CN); Chenghai HU, Hangzhou City (CN)

(21) Appl. No.: 19/538,728

(22) Filed: Feb. 12, 2026

(30) Foreign Application Priority Data
Mar. 20, 2025 (CN) 2025103306074

(51) Int. Cl. F02G 3/00; B01D 53/047; B01D 53/18; B01J 19/00; B01J 19/24; F24F 3/06; H01M 8/04111; H01M 8/0612; H01M 8/0662

(52) U.S. Cl. CPC F02G 3/00; B01D 53/18; B01J 19/0013; B01J 19/245; F24F 3/06; H01M 8/04111; H01M 8/0618; H01M 8/0662; B01D 53/047; B01D 2256/16; B01D 2257/204; B01D 2257/304; B01D 2258/0283; B01J 2219/00103; B01J 2219/00157; H01M 2250/10

(57) ABSTRACT

A comprehensive energy supply system with a hydrogen production prediction and calculation function and integrated with hydrogen energy storage by residual flue gas of a gas engine is provided. When a total amount of discharged flue gas is larger than a first amount of flue gas required by a chiller/heater, remaining flue gas is introduced into the exhaust gas waste heat boiler for generating steam. A second amount of flue gas discharged from the hydrogenation reaction combustion furnace and entering the exhaust gas waste heat boiler is determined so as to calculate a total amount of intake flue gas into the exhaust gas waste heat boiler. A required amount of natural gas raw material is calculated according to the mass flow of steam and a total carbon flow in natural gas, and natural gas is introduced into the furnace according to the amount of natural gas raw material.

### CROSS REFERENCE TO RELATED APPLICATIONS

This application claims priority benefits to CN patent application No. 2025103306074, filed Mar. 20, 2025, the contents of which are incorporated herein by reference.

### TECHNICAL FIELD

The disclosure relates to the technical field of energy supply, in particular to a comprehensive energy supply system with a hydrogen production prediction and calculation function and integrated with hydrogen energy storage by residual flue gas of a gas engine.

### BACKGROUND ART

As clean and carbon-free new energy, hydrogen energy will play an increasingly important role in the field of building in the foreseeable future. Currently, conventional natural-gas reforming hydrogen production is with huge equipment and is widely used in industry. There is still no perfect, feasible and implementable scheme to apply the natural-gas reforming hydrogen production to the field of building.

Meanwhile, an effective recycling mechanism has not yet been formed for residual heat in flue gas generated in a process of power generation by a gas engine, which causes secondary waste of energy and increases embodied carbon emission of such a system. It has become an urgent problem to be solved to effectively combine the power generation by the gas engine, waste heat recovery, hydrogen energy storage, and buildings.

### SUMMARY

In view of shortcomings in related art, a comprehensive energy supply system based on a hydrogen energy storage system using residual flue gas of a gas engine effectively combined with a building energy supply system is provided in the disclosure, which integrates combined cooling, heating and power by a natural-gas reforming hydrogen production device, an exhaust gas waste heat boiler and a gas engine so as to be directly applied to energy supply to buildings and form effective recycling.

The comprehensive energy supply system based on the hydrogen energy storage system using residual flue gas of the gas engine includes the gas engine, a reforming hydrogen production device, and a lithium bromide absorption cold and hot water unit.

An input end of the lithium bromide absorption cold and hot water unit is connected with a high-temperature flue gas channel of the gas engine, and an output end of the lithium bromide absorption cold and hot water unit is connected with a cold and hot water pipeline for air conditioning in a building.

The reforming hydrogen production device includes an exhaust gas waste heat boiler, a hydrogenation reaction combustion furnace, and a medium temperature shift converter. A first input end of the exhaust gas waste heat boiler is connected with the high-temperature flue gas channel of the gas engine, and a second input end of the exhaust gas waste heat boiler is connected with a flue gas channel of the hydrogenation reaction combustion furnace. The medium temperature shift converter is connected at a rear end of the hydrogenation reaction combustion furnace. A terminal hydrogen output end of the reforming hydrogen production device is connected with a hydrogen fuel cell.

A total amount of discharged flue gas by the gas engine per unit time is calculated, and a first amount of flue gas required by the lithium bromide absorption cold and hot water unit per unit time is calculated.

When the total amount of discharged flue gas by the gas engine is less than or equal to the first amount of flue gas required by the lithium bromide absorption cold and hot water unit, all of flue gas is introduced into the lithium bromide absorption cold and hot water unit.

When the total amount of discharged flue gas by the gas engine is larger than the first amount of flue gas required by the lithium bromide absorption cold and hot water unit, the amount of flue gas required by the lithium bromide absorption cold and hot water unit is sufficiently introduced into the lithium bromide absorption cold and hot water unit, and remaining flue gas is introduced into the exhaust gas waste heat boiler for generating steam. A second amount of flue gas discharged from the hydrogenation reaction combustion furnace and entering the exhaust gas waste heat boiler is determined according to an operation state of the hydrogenation reaction combustion furnace. A total amount of intake flue gas into the exhaust gas waste heat boiler is calculated according to the total amount of discharged flue gas, the first amount of flue gas, and the second amount of flue gas.

A mass flow of generated steam is calculated according to the total amount of intake flue gas, a required amount of natural gas raw material is calculated according to the mass flow of steam, and natural gas is introduced into the hydrogenation reaction combustion furnace according to the amount of natural gas raw material.

Beneficial effects are as follows: 1) compared with existing hydrogen production apparatuses using residual heat recycling, some existing apparatuses only realize production of steam through residual heat recovery technology, and then hydrogen can be obtained combined with natural gas for mixed reaction and transformation. An object of the disclosure is to effectively utilize residual heat and residual flue gas of a gas-engine combined cooling, heating and power system to be organically combined with a natural-gas reforming hydrogen production method, and when cold and heat load demand of the building is insufficient in a transitional season, the gas-engine combined cooling, heating and power system can effectively recycle sufficient residual flue gas of the gas engine through the exhaust gas waste heat boiler to prepare high-temperature steam for the natural-gas reforming hydrogen production for hydrogenation reaction. On one hand, hydrogen production cost of a conventional natural-gas reforming hydrogen production method can be effectively reduced, mainly saving cost of steam fuel for hydrogen production to a great extent, and on the other hand, residual heat of residual flue gas of the gas engine can be effectively avoided when the heat load demand of the buildings is small. In utilization of the residual flue gas, in the disclosure, not only high-temperature residual flue gas of the gas engine is utilized, but also the flue gas in the hydrogenation reaction combustion furnace is effectively integrated and utilized, thereby further saving cost of heating raw material in hydrogen production and saving raw material cost by 30% to 50%. In addition, when the energy supply apparatus and method according to the disclosure are applied to actual buildings, hourly hydrogen production amount and required amount of natural gas raw material can be accurately calculated and predicted according to hourly cool, heat and power loads of different buildings with the method according to the disclosure.

2) In a case that the natural-gas reforming hydrogen production can be modularized and miniaturized, with the system proposed in this disclosure, combined cooling, heating and power by the natural-gas reforming hydrogen production device, the exhaust gas waste heat boiler and the gas engine which are modularized and miniaturized can be directly used in buildings after being assembled at a construction site, which has practical engineering application value.

In this disclosure, a hydrogen fuel cell that can effectively utilize hydrogen nearby is selected. When the hydrogen fuel cell is applied in this system, on one hand, power supply to the building can be directly realized, and on the other hand, residual heat of the hydrogen fuel cell can be effectively recycled to generate heat energy to a thermal energy storage tank for the buildings. It is considered in innovative design of the system of the disclosure that economy of the generated hydrogen is poor if it is transported in a long distance, and only when the generated hydrogen is directly used nearby, it produces higher economic benefits. With improvement in structure and method in the disclosure, combined cooling, heating and power by the reforming hydrogen production device, the exhaust gas waste heat boiler and the gas engine can be integrated for comprehensive energy supply for the whole building or building complex.

As a preferred implementation, a method for calculating the required amount of natural gas raw material according to the mass flow of steam and a total carbon flow in natural gas includes:
calculating the total carbon flow in the natural gas according to proportions of respective component gas in the natural gas;
determining a steam-to-carbon ratio in hydrogen production from natural gas, and calculating a volume flow of natural gas according to a calculation formula of the steam-to-carbon ratio combined with the total carbon flow in the natural gas and the mass flow of steam; and
calculating the amount of natural gas raw material to be introduced according to a chemical reaction formula of the hydrogen production from natural gas, combined with the total carbon flow in the natural gas and the mass flow of steam generated by the exhaust gas waste heat boiler.

As a preferred implementation, a method for calculating the total amount of discharged flue gas by the gas engine per unit time includes:
determining hourly power load demand of the building, and calculating actual hourly power generation of the gas engine according to green power generation of the building, power generation of the hydrogen fuel cell and rated power generation of the gas engine under full load in a formula as follows:

$${E}_{F L} \times 50 \% {E}_{L} - {E}_{P} - {E}_{F C} \leq {E}_{F L} \times 50 \%$$

$${P}_{e n g i n e} = {E}_{L} - {E}_{P} - {E}_{F C} {E}_{F L} \times 50 \% < {E}_{P} - {E}_{F C} \leq {E}_{F L}$$

$${E}_{F L} {E}_{L} - {E}_{P} - {E}_{F C} > {E}_{F L}$$

where P_{engine} (kW) is the power generation of the gas engine, E_{FL} (KW) is the rated power generation of the gas engine under full load, E_{L} (KW) is the hourly power load of the building, Eₚ (kW) is the green power generation, and E_{Fc} (KW) is the power generation of the hydrogen fuel cell; and
green power includes solar energy or wind energy connected to the building; and
determining the total amount of discharged flue gas by the gas engine generated with power generation according to the hourly power generation of the gas engine.

As a preferred implementation, a method of calculating the total amount Mₛₘₒₖₑ (kg/h) of intake flue gas into the exhaust gas waste heat boiler according to the total amount of discharged flue gas, the first amount of flue gas, and the second amount of flue gas is shown in a formula as follows:

$${M}_{s m o k e} = \begin{matrix}{M}_{b} & {M}_{T} - {M}_{C / H} < 0 \\ {M}_{T} - {M}_{C / H} + {M}_{b} & {M}_{T} - {M}_{C / H} \geq 0\end{matrix}$$

where M_{T} (kg/h) is the total amount of discharged flue gas by the gas engine per unit time, M_{C/H} (kg/h) is the first amount of flue gas required for cooling or heating of the lithium bromide absorption cold and hot water unit, and M_{b} (kg/h) is the second amount of flue gas discharged from the hydrogenation reaction combustion furnace and entering the exhaust gas waste heat boiler.

As a preferred implementation, the mass flow of generated steam is calculated according to the total amount of intake flue gas, which is realized in a formula as follows:

$${M}_{steam} = \frac{Δ Q}{{h}_{1} - {h}_{2}} = \frac{{C}_{smoke} \times {M}_{s m o k e} \times ( {t}_{1} - {t}_{0} ) \times α}{{h}_{1} - {h}_{2}}$$

Mₛₜₑₐₘ—the mass flow of steam generated by the exhaust gas waste heat boiler (kg/h);
ΔQ—amount of residual heat of flue gas which can be recovered by the exhaust gas waste heat boiler (kJ/h);
h₁—enthalpy of steam in hydrogen reaction (kJ/kg);
h₂—enthalpy of industrial water at room temperature (kJ/kg);
C—specific heat capacity of flue gas (kJ/(kg·° C.));
Mₛₘₒₖₑ—amount of flue gas entering the exhaust gas waste heat boiler (kg/h);
t₁—average temperature of flue gas at an inlet of the exhaust gas waste heat boiler (° C.);
tₒ—average temperature of flue gas at an outlet of the exhaust gas waste heat boiler (° C.); and
α—heat loss coefficient of flue gas.

As a preferred implementation, the calculating the amount of natural gas raw material to be introduced according to the chemical reaction formula of the hydrogen production from natural gas, combined with the total carbon flow in the natural gas and the mass flow of steam generated by the exhaust gas waste heat boiler specifically performed in a formula as follows: The total carbon flow in the natural gas is calculated in a formula as follows:

$$\sum {V}_{c a r b o n} = {γ}_{CH 4} + {γ}_{C 2 H 6} \times 2 + {γ}_{C 3 H 8} \times 3 + {γ}_{C 4 H 10} \times 4 ,$$

where ΣV_{carbon} is the total carbon flow in the natural gas, Γ_{CH4} is a volume percentage of methane in the natural gas, γ_{C2H6} is a volume percentage of ethane, γ_{C3H8} is a volume percentage of propane, and γ_{CAF10} is a volume percentage of butane;

The determining the steam-to-carbon ratio in hydrogen production from natural gas and calculating the volume flow of natural gas according to the calculation formula of the steam-to-carbon ratio combined with the total carbon flow in the natural gas and a volume flow of steam is performed in a formula as follows:

$$ξ = \frac{{V}_{steam}}{{V}_{natural} \times \sum {V}_{carbon}} ,$$

where ξ is the steam-to-carbon ratio, which is empirically determined to be a constant, Vₙₐₜᵤᵣₐₗ is the volume flow of natural gas (Nm³/h), and Vₛₜₑₐₘ is the volume flow of steam (Nm³/h).

As a preferred implementation, the reforming hydrogen production device further includes a natural gas pipeline, a desulfurizer, a mixer for desulfurized natural gas and steam, a gas-liquid separator, a hydrogen purification apparatus, and a waste gas collector.

A first input end of the mixer for desulfurized natural gas and steam is connected with a steam output end of the exhaust gas waste heat boiler, and an output end of the mixer for desulfurized natural gas and steam is connected with the hydrogenation reaction combustion furnace.

A first output end of the natural gas pipeline is connected to the desulfurizer, and an output end of the desulfurizer is connected to a second input end of the mixer for desulfurized natural gas and steam.

The hydrogenation reaction combustion furnace includes a high-temperature reaction furnace and a flue gas channel. An input end of the high-temperature reaction furnace is connected with the output end of the mixer for desulfurized natural gas and steam, and a flue gas output end of the flue gas channel is connected with the exhaust gas waste heat boiler. An output end of the high-temperature reaction furnace is connected with an input end of the medium temperature shift converter, an output end of the medium temperature shift converter is connected with an input end of the gas-liquid separator, and an output end of the gas-liquid separator is connected with an input end of the hydrogen purification apparatus.

A first output end of the hydrogen purification apparatus is connected with a hydrogen energy power generation apparatus, a second output end of the hydrogen purification apparatus is connected with the waste gas collector, and an output end of the waste gas collector is connected with the natural gas pipeline.

As a preferred implementation, the comprehensive energy supply system further includes a hydrogen compressor and a natural gas compressor.

An input end of the hydrogen compressor is connected with the hydrogen purification apparatus, and an output end of the hydrogen compressor is connected with the hydrogen energy power generation apparatus.

An input end of the natural gas compressor is connected with the natural gas pipeline, and an output end of the natural gas compressor is connected with an input end of the desulfurizer.

As a preferred implementation, the comprehensive energy supply system further includes a first heat exchanger, a second heat exchanger and a third heat exchanger. The first heat exchanger is installed in a connecting pipeline between the high-temperature reaction furnace and the mixer for desulfurized natural gas and steam.

The second heat exchanger includes a first cooling channel and a second cooling channel. The first cooling channel is installed in a connecting pipeline between the desulfurizer and the high-temperature reaction furnace, and the second cooling channel is installed in a connecting pipeline between a compressor and the medium temperature shift converter.

The third heat exchanger is installed in a connecting pipeline between the gas-liquid separator and the medium temperature shift converter.

As a preferred implementation, the comprehensive energy supply system further includes a thermal energy storage tank. One end of the third heat exchanger is a cooling water input end, and the other end of the third heat exchanger is output to one of input ends of the thermal storage tank, and another of the input ends of the thermal storage tank is connected with a thermal energy output end of the hydrogen energy power generation apparatus.

### BRIEF DESCRIPTION OF THE DRAWINGS

In order to explain the embodiments of the present disclosure or the technical scheme in the prior art more clearly, the drawings required in the description of the embodiments or the prior art will be briefly introduced below; obviously, the drawings in the following description are only some embodiments of the present disclosure, and other drawings can be obtained according to these drawings by those of ordinary skilled in the art without paying creative labor.

FIG. 1 is an overall structural view of a comprehensive energy supply system based on a hydrogen energy storage system using residual flue gas of a gas engine according to the disclosure;

FIG. 2 is a calculation flow chart of a total carbon flow and hydrogen production from natural gas; and

FIG. 3 is a flow chart of calculation of an amount of natural gas raw material and calculation of hydrogen production.

### DETAILED DESCRIPTION

The present disclosure will be further described in detail with reference to the following examples which present explanation the present disclosure and the present disclosure is not limited to the following examples. Any change or substitution within the technical scope disclosed by the present disclosure should be encompassed within the protection scope of the present disclosure.

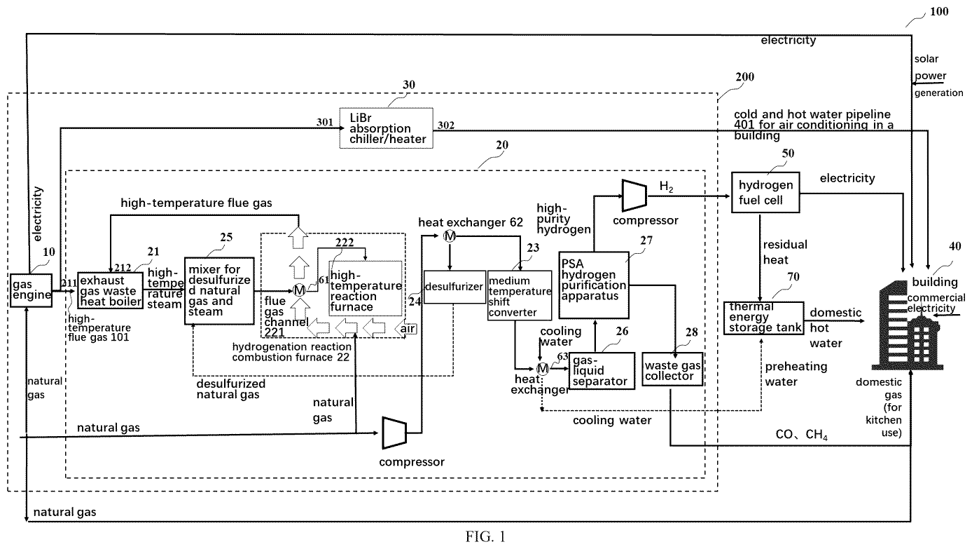

Referring to FIG. 1, a comprehensive energy supply system 100 based on a hydrogen energy storage system 200 using residual flue gas of a gas engine includes a gas engine 10, a reforming hydrogen production device 20, and a lithium bromide absorption cold and hot water unit (can also be called chiller/heater) 30.

An input end 301 of the lithium bromide absorption cold and hot water unit is connected with a high-temperature flue gas channel 101 of the gas engine, and an output end 302 of the lithium bromide absorption cold and hot water unit is connected with a cold and hot water pipeline 401 for air conditioning in a building.

The reforming hydrogen production device 20 includes an exhaust gas waste heat boiler 21, a hydrogenation reaction combustion furnace 22, and a medium temperature shift converter 23. A first input end 211 of the exhaust gas waste heat boiler is connected with the high-temperature flue gas channel of the gas engine, and a second input end 212 of the exhaust gas waste heat boiler is connected with a flue gas channel 221 of the hydrogenation reaction combustion furnace. The medium temperature shift converter is connected at a rear end of the hydrogenation reaction combustion furnace. A terminal hydrogen output end of the reforming hydrogen production device is connected with a hydrogen fuel cell 50.

The comprehensive energy supply system is configured to perform the following steps:
determine a calculated total amount of discharged flue gas by the gas engine per unit time and a calculated first amount of flue gas required by the lithium bromide absorption cold and hot water unit per unit time, introduce all of flue gas into the lithium bromide absorption cold and hot water unit when the total amount of discharged flue gas by the gas engine is less than or equal to the first amount of flue gas required by the lithium bromide absorption cold and hot water unit; sufficiently introduce the amount of flue gas required by the lithium bromide absorption cold and hot water unit into the lithium bromide absorption cold and hot water unit and introduce remaining flue gas into the exhaust gas waste heat boiler for generating steam when the total amount of discharged flue gas by the gas engine is larger than the first amount of flue gas required by the lithium bromide absorption cold and hot water unit;
determine a second amount of flue gas discharged from the hydrogenation reaction combustion furnace and entering the exhaust gas waste heat boiler according to an operation state of the hydrogenation reaction combustion furnace; and calculate a total amount of intake flue gas into the exhaust gas waste heat boiler according to the total amount of discharged flue gas, the first amount of flue gas, and the second amount of flue gas; and
calculate a mass flow of generated steam according to the total amount of intake flue gas, calculate hydrogen production according to the mass flow of steam, calculate a required amount of natural gas raw material according to the hydrogen production, and introduce natural gas into the hydrogenation reaction combustion furnace according to the amount of natural gas raw material.

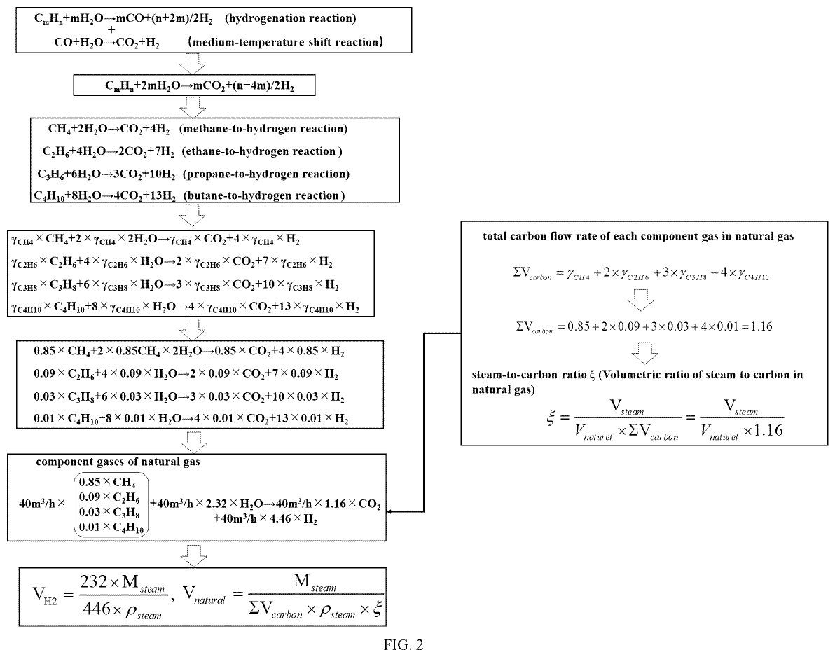

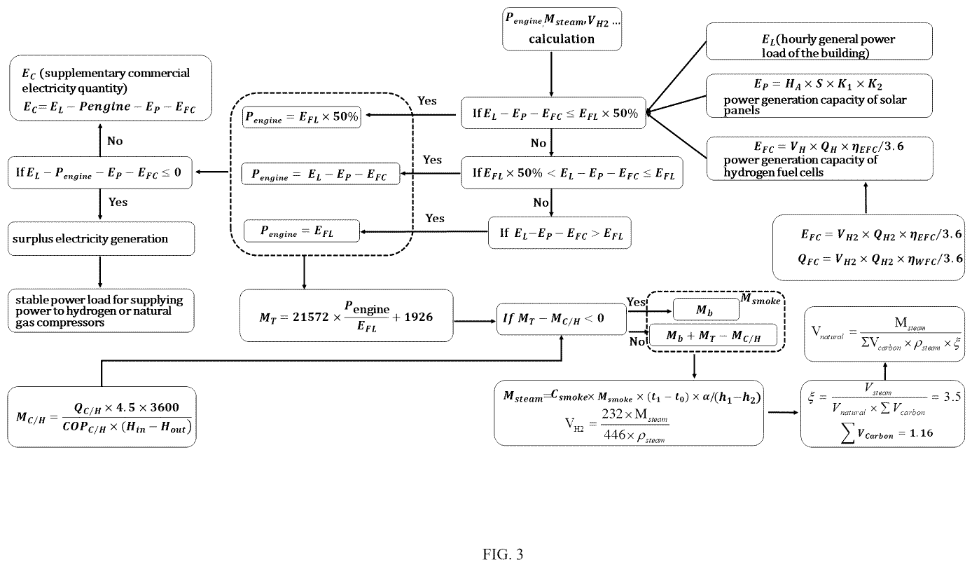

The disclosure following is illustrated with reference to FIGS. 2 and 3.

(1) The total amount of discharged flue gas by the gas engine per unit time is calculated in a formula as follows:

The gas engine can use the natural gas to generate power and high-temperature flue gas at the same time. Power generation and heat are calculated as follows:

$${P}_{engine} = {V}_{engine} \times {Q}_{gas} \times {η}_{engine} / 3.6 \qquad ( 1 )$$

$${Q}_{engine} = \frac{{P}_{engine}}{{η}_{engine}} ( 1 - {η}_{engine} - {η}_{L} ) \qquad ( 2 )$$

where P_{engine} (kW) is power generation of the gas engine, V_{engine} (m³/h) is natural gas consumption of the gas engine, Q_{gas} (MJ/m³) is a calorific value of natural gas, η_{engine} is power generation efficiency of the gas engine, and Q_{engine} (kW) is heat recovered by the high-temperature residual flue gas of the gas engine through the exhaust gas waste heat boiler, and η_{L} is a heat loss rate of the gas engine.

Specifically, a method for calculating the total amount of discharged flue gas by the gas engine per unit time includes determining hourly power load demand of the building 40, calculating actual hourly power generation of the gas engine according to green power generation of the building, power generation of the hydrogen fuel cell and rated power generation of the gas engine under full load, and then determining the total amount M_{T} (kg/h) of flue gas generated with power generation according to the hourly power generation of the gas engine.

Green power includes solar energy or wind energy connected to the building.

The hourly power generation of the gas engine is calculated specifically as follows:

First of all, power generation P_{engine} (kwh/h) of the gas engine is based on performance of the device and a power supply configuration method set in this disclosure, that is, a load rate of the gas engine should be maintained at 50% to 100% for safe operation. After the gas engine supplies power to the building, the building can be supplied by solar energy and wind energy, and if power is still insufficient, the building can be supplied by commercial power. Therefore, the power generation P_{engine} (kWh/h) of the gas engine can be inferred and determined in a formula as follows:

$${E}_{F L} \times 50 \% {E}_{L} - {E}_{P} - {E}_{F C} \leq {E}_{F L} \times 50 \% \qquad ( 3 )$$

$${P}_{e n g i n e} = {E}_{L} - {E}_{P} - {E}_{F C} {E}_{F L} \times 50 \% < {E}_{P} - {E}_{F C} \leq {E}_{F L}$$

$${E}_{F L} {E}_{L} - {E}_{P} - {E}_{F C} > {E}_{F L}$$

where P_{engine} (kW) is the power generation of the gas engine, E_{FL} (KW) is the rated power generation of the gas engine under full load, E_{L} (KW) is the hourly power load of the building, E_{P} (kW) is the green power generation, and E_{FC} (kW) is the power generation of the hydrogen fuel cell. For safe operation of the gas engine, its operating load generally needs to be maintained above 50%. When the power generation of the gas engine is known, the amount of natural gas consumed by the gas engine can be calculated by using the formula (1) described above.

Specifically, taking the green power being solar power generation as an example, renewable energy solar power generation E_{P} (kW) is preferentially used in building power supply. Because the load rate of the gas engine needs to be maintained above 50%, when E_{L}−E_{P}−E_{FC}≤E_{FL}×50%, the power generation of the gas engine is E_{FL}×50%; when E_{FL}×50%<E_{L}−E_{P}−E_{FC}≤E_{FL}, the power generation of the gas engines is E_{L}−E_{P}−E_{FC}; and when E_{L}−E_{P}−E_{FC}>E_{FL}, the power generation of the gas engine is a total rated power generation E_{FL} of the gas engine. When E_{L}−Pengine−E_{P}−E_{FC}≤0, that is, when a sum of the power generation of the gas engine, solar power generation and the power generation of the hydrogen fuel cell cannot cover the power load demand of the building, insufficient power is all supplemented by commercial power EC. If there is surplus power, all of the surplus power is used to provide stable power load required by the hydrogen or natural gas compressor to compress gas. The solar power generation and the power generation of the hydrogen fuel cell can be calculated in corresponding formulas (5) and (6), and hourly power load E_{L} (KW) of the building can be obtained by combining historical building data and survey data.

A method of calculating the total amount of discharged flue gas by the gas engine according to the power generation of the gas engine is as follows. According to analysis of performance data of the gas engine provided by an equipment manufacturer, the amount M_{T} (kg/h) of the flue gas of the gas engine is calculated as shown in formula (4).

$${M}_{T} = 215 \; 72 \times φ + 1926 = 21572 \times \frac{{P}_{engine}}{{E}_{T G E}} + 1926 \qquad ( 4 )$$

where M_{T} (kg/h) is the total amount of discharged flue gas by the gas engine, and φ is the load rate of the gas engine, which is a ratio of actual power generation to rated full-load power generation of the gas engine, and E_{TGE} (KW) is the rated full-load power generation.

Regarding green power, taking solar panels as an example in the disclosure, the solar power generation is calculated in a formula as follows:

$${E}_{P} = {H}_{A} \times S \times {K}_{1} \times {K}_{2} \qquad ( 5 )$$

where Eₚ is power generation of solar panels (kW), H_{A} is solar radiation per unit area (kW/m²), S is the area of solar panels (m2), and K₁ is module conversion efficiency (generally 16 to 18%, 17% in this disclosure); and K₂ is overall efficiency of a system (generally 75% to 85%, 80% in this disclosure). In addition, H_{A} (kW/m²) data comes from climate data obtained from survey.

The power generation of the hydrogen fuel cell is specifically calculated as follows in formula (6) and formula (7):

$${E}_{F C} = {V}_{H 2} \times {Q}_{H 2} \times {η}_{EFC} / 3.6 \qquad ( 6 )$$

$${Q}_{F C} = {V}_{H 2} \times {Q}_{H 2} \times {η}_{WFC} / 3.6 \qquad ( 7 )$$

where E_{FC} (W) is the power generation of the hydrogen fuel cell, V_{H2} (Nm³/h) is amount of hydrogen entering the hydrogen fuel cell for power generation, Q_{H2} (MJ/m³) is a calorific value of hydrogen, ηE_{FC} is power generation efficiency of the hydrogen fuel cell, and η_{WFC} is heat recovery efficiency of the hydrogen fuel cell.

The total amount Mₛₘₒₖₑ (kg/h) of intake flue gas into the exhaust gas waste heat boiler are calculated according to the total amount M_{T} (kg/h) of discharged flue gas by the gas engine, the first amount of flue gas M_{C/H} (kg/h) and the second amount of flue gas M_{b} (kg/h), and Mₛₘₒₖₑ is calculated in formula (8) as follows:

$${M}_{s m o k e} = \begin{matrix}{M}_{b} & {M}_{T} - {M}_{C / H} < 0 \\ {M}_{T} - {M}_{C / H} + {M}_{b} & {M}_{T} - {M}_{C / H} \geq 0\end{matrix} \qquad ( 8 )$$

where M_{T} (kg/h) is the total amount of discharged flue gas by the gas engine, M_{C/H} (kg/h) is the first amount of flue gas required for cooling or heating of the lithium bromide absorption cold and hot water unit, and M_{b} (kg/h) is the second amount of flue gas discharged from the hydrogenation reaction combustion furnace and entering the exhaust gas waste heat boiler.

When there is cooling and heating load demand in the building, a part of the flue gas of the gas engine enters the lithium bromide absorption cold and hot water unit to generate cold and hot water for air conditioning in the building, that is, the first amount of flue gas M_{C/H} (kg/h) and all of rest of the flue gas of the gas engine M_{T}−M_{C/H} (kg/h) enters the exhaust gas waste heat boiler to generate steam for hydrogen production.

The total amount Mₛₘₒₖₑ (kg/h) of intake flue gas entering the exhaust gas waste heat boiler for generating steam is not only from the gas engine, but also contains the second amount of flue gas M_{b} (kg/h) discharged from the hydrogenation reaction combustion furnace and entering the exhaust gas waste heat boiler. When M_{T}−M_{C/H}<0, it indicates that the amount of flue gas generated by the gas engine is only enough to meet cooling and heating load demand for air conditioning in the building. At this time, the amount of flue gas entering the exhaust gas waste heat boiler to generate steam for hydrogen production only involves M_{b} (kg/h) from a hydrogenation reactor. When M_{T}−M_{C/H}≥0, it indicates that the flue gas generated by the gas engine is not only used to provide the cooling and heating load demand for the building, but still with remaining flue gas, and at this time, the total amount Mₛₘₒₖₑ (kg/h) of intake flue gas into the exhaust gas waste heat boiler is M_{T}−M_{C/H}+M_{b}. If a hydrogenation reaction burner is currently in operation, the total amount of intake flue gas includes the second amount of flue gas M_{b}, and if the hydrogenation reaction burner is currently not in operation, the second amount of flue gas M_{b} is 0.

More specifically, the first amount of flue gas required for preparing cold and hot water for air conditioning in the building by the lithium bromide absorption cold and hot water unit can be calculated in formula (9) as follows:

$${M}_{C / H} = \frac{{Q}_{C / H} \times 4.5 \times 3600}{{COP}_{C / H} \times ( {h}_{i n} - {h}_{o u t} )} \qquad ( 9 )$$

where M_{C/H} (kg/h) is an amount of flue gas required for cooling or heating by the lithium bromide absorption cold and hot water unit, hᵢₙ (kJ/kg) is enthalpy of flue gas entering the lithium bromide absorption cold and hot water unit, hₒᵤₜ (kJ/kg) is enthalpy of flue gas leaving the lithium bromide absorption cold and hot water unit, COP_{C/H} is refrigeration and heating performance coefficient (COP) of the lithium bromide absorption cold and hot water unit, which is a constant. Q_{C/H} (kWh/h) is hourly cooling load or heating load of different types of buildings. With investigation, the hourly cooling load or heating load value of different types of buildings can be obtained, which is a product of daily cooling load or heating load value and percentage of hourly load, where hᵢₙ (kJ/kg) and hₒᵤₜ (kJ/kg) are calculated in formula (10) as follows:

$${h}_{S} = 11.157 \times t - 306.2 \qquad ( 10 )$$

where h_{S} (kJ/kg) is enthalpy of flue gas and t (° C.) is temperature of the flue gas. This formula is summarized by the applicant based on an enthalpy table of flue gas of the gas engine residual, and hᵢₙ and hₒᵤₜ can be determined by substituting temperature of flue gas when entering and temperature of flue gas when leaving into the formula (10).

(2) A mass flow of generated steam is calculated according to the total amount of intake flue gas, a required amount of natural gas raw material is calculated according to the mass flow of steam and the total carbon flow in the natural gas, and natural gas is introduced into the hydrogenation reaction combustion furnace according to the amount of natural gas raw material.

When the amount of flue gas Mₛₘₒₖₑ (kg/h) entering the exhaust gas waste heat boiler is determined, the mass flow Mₛₜₑₐₘ (kg/h) of steam for hydrogen production generated by the exhaust gas waste heat boiler accordingly can be determined according to formula (11):

$${M}_{s t e a m} = \frac{Δ Q}{{h}_{1} - {h}_{2}} = \frac{{C}_{s m o k e} \times {M}_{s m o k e} \times ( {t}_{1} - {t}_{0} ) \times α}{{h}_{1} - {h}_{2}} \qquad ( 11 )$$

Mₛₜₑₐₘ—the mass flow of steam generated by the exhaust gas waste heat boiler (kg/h);
ΔQ—amount of residual heat of flue gas which can be recovered by the exhaust gas waste heat boiler (kJ/h);
h₁—enthalpy of steam in hydrogen reaction (kJ/kg);
h₂—enthalpy of industrial water at room temperature (kJ/kg), which can be illustratively set to 20° C. at room temperature;
C—specific heat capacity of flue gas (kJ/(kg·° C.)), which is illustratively taken as 1.15 kJ/(kg·° C.);
Mₛₘₒₖₑ—amount of flue gas entering the exhaust gas waste heat boiler (kg/h);
t₁—average temperature of flue gas at an inlet of the exhaust gas waste heat boiler (° C.);
tₒ—average temperature of flue gas at an outlet of the exhaust gas waste heat boiler (° C.), which is generally taken as 150° C.; and
α—heat loss coefficient of flue gas, which is usually taken as 0.9.

According to actual situation of equipment performance, exhaust temperature of the exhaust gas waste heat boiler should generally not be lower than 150° C., otherwise a tail heated surface may be corroded. The amount ΔQ of residual heat of flue gas which can be recovered by the exhaust gas waste heat boiler is shown in formula (12) as follows:

$$Δ Q = {C}_{smoke} \times {M}_{s moke} \times ( {t}_{1} - {t}_{0} ) \times α \qquad ( 12 )$$

where ΔQ indicates residual heat of flue gas recovered by the exhaust gas waste heat boiler (kJ/h), Cₛₘₒₖₑ is specific heat capacity of flue gas (kJ/(kg° C.)), with a value of 1.15 kJ/(kg·° C.), Mₛₘₒₖₑ is the mass flow of flue gas entering the exhaust gas waste heat boiler (kg/h), t₁ the average temperature of flue gas at an inlet of the exhaust gas waste heat boiler (° C.), tₒ the average temperature of flue gas at an outlet of the exhaust gas waste heat boiler (° C.), which is generally taken as 150° C., and the exhaust temperature of the exhaust gas waste heat boiler should generally not be lower than 150° C., otherwise a tail heated surface may be corroded; and α is the heat loss coefficient of flue gas, which is usually taken as 0.9.

Then, a hydrogen production amount V_{H2} (Nm³/h) that can be generated by a corresponding mass flow Mₛₜₑₐₘ (kg/h) of steam for hydrogen production is determined in formula (13) as follows:

$${V}_{H 2} = \frac{232 \times {M}_{s t e a m}}{446 \times {ρ}_{s t e a m}} \qquad ( 13 )$$

where V_{H2} (Nm³/h) is an actual hydrogen production amount; Mₛₜₑₐₘ (kg/h) is the mass flow of steam accordingly generated by the exhaust gas waste heat boiler for heating water when the residual flue gas recovered, and ρₛₜₑₐₘ is density of steam (kg/Nm³). It should be specially noted that the hydrogen production amount in this disclosure is determined by the amount of residual heat recovered by the exhaust gas waste heat boiler, that is, hydrogen is determined by heat. When there is no residual flue gas from the gas engine entering the exhaust gas waste heat boiler to generate steam required for hydrogenation reaction, the hydrogen production amount is correspondingly zero. Hydrogen production can be carried out only when there is residual heat of residual flue gas being used to generate steam required for hydrogenation reaction, otherwise hydrogen production stops. This hydrogen production method can be used to calculate the power generation of the hydrogen fuel cell.

Main reactions in a process of hydrogen production are in formula (14) to formula (16) as follows:

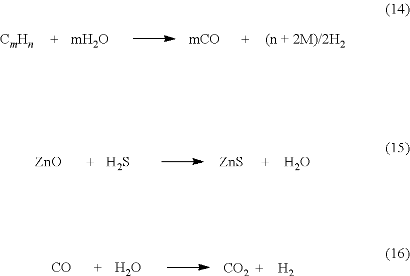

A calculation process of hydrogen production amount from the flue gas of the gas engine and the carbon flow is as shown in FIG. 2. The natural gas mainly includes four types of gas, each of which can produce hydrogen. In a first stage, reactions of the four types of gas follow a chemical formula as follows: CₘHₙ+mH₂O→mCO+ (n+2m)/2H₂, which is an endothermic reaction. In a medium temperature shift reaction in a second stage, the reactions follow a formula as follows: CO+H₂O→CO₂+H₂, which is an exothermic reaction. To sum up, two-stage chemical reactions of each gas in the natural gas is: CₘHₙ+2mH₂O→mCO₂+(4m+n)/2H₂, where m can be 1, 2, 3 and 4, and n can be taken as 4, 6, 8 and 10 accordingly, which respectively represent four different types of gas in the natural gas. Main components of the natural gas is methane (CH₄), accounting for 85% by volume, followed by ethane (C₂H₆) accounting for 9%, propane (C₃H₈) accounting for 3%, and butane (C₄H₁₀) accounting for 1%. In addition, the natural gas also contains 2% nitrogen (N₂) and a trace amount of hydrogen sulfide (H₂S).

Based on composition of the natural gas, a calculation formula of carbon atoms in the natural gas obtained in this disclosure is shown in formula (17):

$$\sum {V}_{c a r b o n} = {γ}_{C H 4} + {γ}_{C 2 H 6} \times 2 + {γ}_{C 3 H 8} \times 3 + {γ}_{C 4 H 10} \times 4 \qquad ( 17 )$$

where ΣV_{carbon} is total carbon atom content of the natural gas, γ_{CH4} is a volume percentage of methane (CH₄) in the natural gas, γ_{C2H6} is a volume percentage of ethane (C₂H₆), γ_{C3H8} is a volume percentage of propane (C₃H₈), and γ_{C4H10} is a volume percentage of butane (C₄H₁₀);

A carbon flow per unit of natural gas can be obtained by multiplying a number of carbon atoms in each gas by a corresponding volume ratio. There is one carbon atom in CH₄, two carbon atoms in C₂H₆, three carbon atoms in C₃H₈ and four carbon atoms in C₄H₁₀, and thus the carbon flow per unit of natural gas is finally calculated to be 1.16. If there is 1 unit volume of natural gas, it contains 0.85 unit volume of methane, and the reaction requires 2×0.85 unit volume of steam, corresponding to 0.85 unit volume of CO₂ and 4×0.85 unit volume of H₂. Calculation processes of hydrogenation reactions of other gas is also shown in FIG. 2. A unit volume of natural gas needs 2×0.85+4×0.09+6×0.03+8×0.01=2.32 unit volume of steam for reaction, which correspondingly produces 4×0.85+7×0.09+10×0.03+13×0.01=4.46 unit volume of hydrogen. Therefore, 40×4.46=178.4 m³/h of H₂ can be output with 40 m³/h of natural gas.

According to the formula (17), a total number of carbon atoms of natural gas is 1.16. Carbon atom content in the natural gas is directly related to an amount of steam required for hydrogenation reaction, and the amount of the residual heat of the flue gas entering the exhaust gas waste heat boiler directly determines the amount of steam generated.

In calculating a mass flow of steam required for hydrogen production by hydrogenation reaction, it is necessary to determine a total carbon atom content in the natural gas to calculate the steam-to-carbon ratio. Under a normal production condition, it is the key in hydrogen production from natural gas to control the steam-to-carbon ratio of water and natural gas. The steam-to-carbon ratio for hydrogen production is generally 3 to 5, and a normal steam-to-carbon ratio is with a value of 3.5. If the steam-to-carbon ratio deviates too much from this ratio, it may result in waste of steam, and if the steam-to-carbon ratio is too small, it may result in carbon deposition on hydrogenation reaction catalyst and deactivation of the hydrogenation reaction catalyst. According to previous researches, the steam-to-carbon ratio of this disclosure is set to 3.5. A calculation formula of the steam-to-carbon ratio is formula (18), and the carbon flow is determined by superposition of respective gas containing carbon atoms in the natural gas:

$$ξ = \frac{{V}_{s t e a m}}{{V}_{n a t u r a l} \times \sum {V}_{c a r b o n}} = 3.5 \qquad ( 18 )$$

where ξ is the steam-to-carbon ratio, which is taken as 3.5 in this disclosure, Vₙₐₜᵤᵣₐₗ is the volume flow of natural gas (Nm³/h), Vₛₜₑₐₘ is the volume flow of steam (Nm³/h), ΣV_{carbon} is a total volume flow of carbon in the natural gas (Nm³/h), and the calculated total number of carbon atoms in the natural gas is 1.16.

According to the formula (17) above, the total number of carbon atoms in the natural gas is 1.16. According to the above formula, the required volume flow of natural gas can be calculated by the volume flow of steam.

Illustratively, if 40 Nm³/h of natural gas is used as raw material to produce hydrogen, the carbon flow of natural gas is a product of the volume flow and the total number of carbon atoms, which is obtained as 46.4 Nm³/h. Since the steam-to-carbon ratio is generally selected as 3.5, a steam flow required for hydrogenation reaction of 40 Nm³/h of natural gas is 162.4 Nm³/h.

The reforming hydrogen production device further includes a natural gas pipeline, a desulfurizer 24, a mixer for desulfurized natural gas and steam 25, a gas-liquid separator 26, a hydrogen purification apparatus 27, and a waste gas collector 28, as shown in FIG. 1.

A first input end of the mixer for desulfurized natural gas and steam is connected with a steam output end of the exhaust gas waste heat boiler, and an output end of the mixer for desulfurized natural gas and steam is connected with the hydrogenation reaction combustion furnace.

A first output end of the natural gas pipeline is connected to the desulfurizer 24, and an output end of the desulfurizer is connected to a second input end of the mixer for desulfurized natural gas and steam 25.

The hydrogenation reaction combustion furnace 22 includes a high-temperature reaction furnace 222 and a flue gas channel 221. An input end of the high-temperature reaction furnace is connected with the output end of the mixer for desulfurized natural gas and steam, and a flue gas output end of the flue gas channel is connected with the exhaust gas waste heat boiler. An output end of the high-temperature reaction furnace is connected with an input end of the medium temperature shift converter, an output end of the medium temperature shift converter is connected with an input end of the gas-liquid separator, and an output end of the gas-liquid separator is connected with an input end of the hydrogen purification apparatus.

A first output end of the hydrogen purification apparatus is connected with a hydrogen energy power generation apparatus, a second output end of the hydrogen purification apparatus is connected with the waste gas collector, and an output end of the waste gas collector is connected with the natural gas pipeline.

The above structure innovatively incorporates residual combustible gas after hydrogen purification in the natural-gas reforming hydrogen production into a building gas supply pipeline, and further effectively combines the residual flue gas hydrogen storage method in this disclosure with energy supply to the building.

In a preferred structure, the comprehensive energy supply system further includes a hydrogen compressor and a natural gas compressor.

An input end of the hydrogen compressor is connected with the hydrogen purification apparatus, and an output end of the hydrogen compressor is connected with the hydrogen energy power generation apparatus.

An input end of the natural gas compressor is connected with the natural gas pipeline, and an output end of the natural gas compressor is connected with an input end of the desulfurizer.

In a preferred structure, the comprehensive energy supply system further includes a first heat exchanger 61, a second heat exchanger 62 and a third heat exchanger 63. The first heat exchanger is installed in a connecting pipeline between the high-temperature reaction furnace and the mixer for desulfurized natural gas and steam.

The second heat exchanger 62 includes a first cooling channel and a second cooling channel. The first cooling channel is installed in a connecting pipeline between the desulfurizer and the high-temperature reaction furnace, and the second cooling channel is installed in a connecting pipeline between a compressor and the medium temperature shift converter.

The third heat exchanger 63 is installed in a connecting pipeline between the gas-liquid separator and the medium temperature shift converter.

The comprehensive energy supply system further includes a thermal energy storage tank 70. One end of the third heat exchanger is a cooling water input end, and the other end of the third heat exchanger is output to one of input ends of the thermal storage tank, and another of the input ends of the thermal storage tank is connected with a thermal energy output end of the hydrogen energy power generation apparatus.

In the above, a specific operation mode of the comprehensive energy supply system is as follows.

The gas engine uses the natural gas as raw material to generate power, while generates high-temperature flue gas at the same time, and temperature of the flue gas can generally reach about 368 to 469° C.

In summer and winter, when the building has stable cooling and heating load demand, the flue gas of the gas engine can be used to generate chilled water/hot water for air conditioning in the building through the lithium bromide absorption cold and hot water unit so as to provide the cooling and heating load demand for the building. When there is no stable cooling and heating load demand in the building during a transition season, the flue gas of the gas engine can pass through the exhaust gas waste heat boiler to generate high-temperature steam required for hydrogen production reaction, thus avoiding waste of residual heat of the flue gas. The high-temperature steam generated by the exhaust gas waste heat boiler and the natural gas desulfurized by the desulfurizer are mixed in a mixer of natural gas and steam, which then together enter the high-temperature reaction furnace in the combustion furnace together for hydrogenation reaction. The natural gas is desulfurized because it contains sulfide, which may make the hydrogenation reaction catalyst ineffective. Therefore, the natural gas in the natural gas pipeline is compressed by the compressor and then needs to be preheated by the heat exchanger before entering the desulfurizer for desulfurization.

The first heat exchanger is built in an inner wall side of the flue gas channel in the combustion furnace, and a mixture of the desulfurized natural gas and the steam first flows through the first heat exchanger in the combustion furnace for preheating, so as to increase a hydrogenation reaction speed and required reaction temperature. The hydrogenation reaction temperature in the high-temperature reactor is about 735° C., and thus it is necessary to use the natural gas in the natural gas pipeline as heating fuel for combustion to increase temperature. The natural gas in the natural gas pipeline burns with oxygen in the air to heat the high-temperature reaction furnace, and meanwhile, flue gas generated by combustion flows through the flue gas channel in the combustion furnace and finally enters the exhaust gas waste heat boiler to generate high-temperature steam for hydrogen production.

A main component of the natural gas is CH₄, and a main chemical equation of its hydrogenation reaction is CH₄+H₂O→CO+3H₂, which is an endothermic process with a reaction temperature of about 735° C. The mixed gas after hydrogenation reaction comes out of the high-temperature reaction furnace and flows through the second heat exchanger to heat the natural gas to be desulfurized. Because desulfurization reaction temperature is between 290° C. and 350° C., the natural gas to be desulfurized needs to be preheated first. However, the mixed gas entering the medium temperature shift converter needs to be cooled down, because the medium temperature shift reaction is an exothermic reaction, with reaction temperature only about 200 to 300° C., and with a main chemical reaction equation of CO+H₂O→CO₂+H₂. The mixed gas after the medium temperature shift reaction comes out of the medium temperature shift converter and flows through the third heat exchanger, and enters the gas-liquid separator with reduced temperature for gas-liquid separation after being cooled by cooling water. After the cooling water is heated by the heat exchanger, it can be stored in the thermal storage tank as domestic hot water for the building, which can also effectively become energy needed by the building. After gas-liquid separation, the steam in the mixed gas is liquefied into water droplets and placed at a bottom of the gas-liquid separator, and gas such as CO₂, H₂, CO and CH₄ rise to a PSA (Pressure Swing Adsorption) hydrogen purification apparatus. Hydrogen is separated from other gas in a PSA hydrogen compression adsorption purification device, and its purity becomes 99.999 Vol %. Other gas in the mixed gas mainly include CO and CO₂, which can be stored in an exhaust gas collector. Combustible gas CO and a small amount of CH₄ gas in the exhaust gas collector can be mixed in the natural gas pipeline and together transported to the building as domestic gas, which makes best use of exhaust gas. Hydrogen purified by the PSA hydrogen purification apparatus can be directly used as raw material of the hydrogen fuel cell apparatus after being compressed by the hydrogen compressor to a certain extent. The hydrogen fuel cell generates power to be supplied to the building, and residual heat generated therewith can be used as a heat source of the thermal storage water tank to heat domestic hot water for the building.

Compared with existing hydrogen production apparatuses using residual heat recycling, some existing apparatuses only realize production of steam through residual heat recovery technology, and then hydrogen can be obtained combined with natural gas for mixed reaction and transformation. An object of the present disclosure is to realize a gas-engine combined cooling, heating and power supply apparatus based on basic technology of power supply by gas engine. In a mature state of hardware technology, a volume of the natural-gas reforming hydrogen production device is reduced, which can be directly incorporated and applied in the building, and the residual flue gas of the gas engine can be effectively utilized, including direct application of hydrogen, hot water of the heat exchanger generated by hydrogen production, the exhaust gas of the exhaust gas collector, etc., so as to maximize energy utilization.

What is claimed is:

1. A comprehensive energy supply system based on a hydrogen energy storage system using residual flue gas of a gas engine, comprising the gas engine, a reforming hydrogen production device, and a lithium bromide absorption cold and hot water unit, wherein
an input end of the lithium bromide absorption cold and hot water unit is connected with a high-temperature flue gas channel of the gas engine, and an output end of the lithium bromide absorption cold and hot water unit is connected with a cold and hot water pipeline for air conditioning in a building;
the reforming hydrogen production device comprises an exhaust gas waste heat boiler, a hydrogenation reaction combustion furnace, and a medium temperature shift converter, a first input end of the exhaust gas waste heat boiler is connected with the high-temperature flue gas channel of the gas engine, and a second input end of the exhaust gas waste heat boiler is connected with a flue gas channel of the hydrogenation reaction combustion furnace, the medium temperature shift converter is connected at a rear end of the hydrogenation reaction combustion furnace, and a terminal hydrogen output end of the reforming hydrogen production device is connected with a hydrogen fuel cell;
a total amount of discharged flue gas by the gas engine per unit time is calculated, and a first amount of flue gas required by the lithium bromide absorption cold and hot water unit per unit time is calculated;
when the total amount of discharged flue gas by the gas engine is less than or equal to the first amount of flue gas required by the lithium bromide absorption cold and hot water unit, all of flue gas is introduced into the lithium bromide absorption cold and hot water unit;
when the total amount of discharged flue gas by the gas engine is larger than the first amount of flue gas required by the lithium bromide absorption cold and hot water unit, the amount of flue gas required by the lithium bromide absorption cold and hot water unit is sufficiently introduced into the lithium bromide absorption cold and hot water unit, and remaining flue gas is introduced into the exhaust gas waste heat boiler for generating steam; a second amount of flue gas discharged from the hydrogenation reaction combustion furnace and entering the exhaust gas waste heat boiler is determined according to an operation state of the hydrogenation reaction combustion furnace; and a total amount of intake flue gas into the exhaust gas waste heat boiler is calculated according to the total amount of discharged flue gas, the first amount of flue gas, and the second amount of flue gas; and
a mass flow of generated steam is calculated according to the total amount of intake flue gas, a required amount of natural gas raw material is calculated according to a mass flow of steam and a total carbon flow in the natural gas, and natural gas is introduced into the hydrogenation reaction combustion furnace according to the amount of natural gas raw material.

2. The comprehensive energy supply system based on a hydrogen energy storage system using residual flue gas of a gas engine according to claim 1, wherein a method for calculating the required amount of natural gas raw material according to the mass flow of steam and the total carbon flow in the natural gas comprises:
calculating the total carbon flow in the natural gas according to proportions of respective component gas in the natural gas;
determining a steam-to-carbon ratio in hydrogen production from natural gas, and calculating a volume flow of natural gas according to a calculation formula of the steam-to-carbon ratio combined with the total carbon flow in the natural gas and the mass flow of steam; and
calculating the amount of natural gas raw material to be introduced according to a chemical reaction formula of the hydrogen production from natural gas, combined with the total carbon flow in the natural gas and the mass flow of steam generated by the exhaust gas waste heat boiler.

3. The comprehensive energy supply system based on a hydrogen energy storage system using residual flue gas of a gas engine according to claim 1, wherein a method for calculating the total amount of discharged flue gas by the gas engine per unit time comprises:
determining hourly power load demand of the building, and calculating actual hourly power generation of the gas engine according to green power generation of the building, power generation of the hydrogen fuel cell and rated power generation of the gas engine under full load in a formula as follows:
$${E}_{FL} \times 50 \% {E}_{L} - {E}_{P} - {E}_{FC} \leq {E}_{F L} \times 50 \%$$
$${P}_{e n g i n e} = {E}_{L} - {E}_{P} - {E}_{FC} {E}_{FL} \times 50 \% < {E}_{L} - {E}_{P} - {E}_{FC} \leq {E}_{FL}$$
$${E}_{FL} {E}_{L} - {E}_{P} - {E}_{FC} > {E}_{FL}$$
where P_{engine} is the power generation of the gas engine in kW, E_{FL} is the rated power generation of the gas engine under full load in kW, E_{L} is the hourly power load of the building in kW, E_{P} is the green power generation in kW, and E_{FC} is the power generation of the hydrogen fuel cell in kW;
wherein green power comprises solar energy or wind energy connected to the building; and
determining the total amount of discharged flue gas by the gas engine generated with power generation according to the hourly power generation of the gas engine.

4. The comprehensive energy supply system based on a hydrogen energy storage system using residual flue gas of a gas engine according to claim 3, wherein a method of calculating the total amount Mₛₘₒₖₑ of intake flue gas into the exhaust gas waste heat boiler in kg/h according to the total amount of discharged flue gas, the first amount of flue gas, and the second amount of flue gas is shown in a formula as follows:
$${M}_{s m o k e} = \begin{matrix}{M}_{b} & {M}_{T} - {M}_{C / H} < 0 \\ {M}_{T} - {M}_{C / H} + {M}_{b} & {M}_{T} - {M}_{C / H} \geq 0\end{matrix}$$
where M_{T} is the total amount of discharged flue gas by the gas engine per unit time in kg/h, M_{C/H} is the first amount of flue gas required for cold or hot water required for cooling or heating of the building generated by the lithium bromide absorption cold and hot water unit in kg/h, and M_{b} is the second amount of flue gas discharged from the hydrogenation reaction combustion furnace and entering the exhaust gas waste heat boiler in kg/h.

5. The comprehensive energy supply system based on a hydrogen energy storage system using residual flue gas of a gas engine according to claim 4, wherein the mass flow of generated steam is calculated according to the total amount of intake flue gas, which is realized in a formula as follows:
$${M}_{steam} = \frac{Δ Q}{{h}_{1} - {h}_{2}} = \frac{{C}_{s m o k e} \times {M}_{s m o k e} \times ( {t}_{1} - {t}_{0} ) \times α}{{h}_{1} - {h}_{2}}$$
Mₛₜₑₐₘ—the mass flow of steam generated by the exhaust gas waste heat boiler in kg/h;
ΔQ—amount of residual heat of flue gas which can be recovered by the exhaust gas waste heat boiler in kJ/h;
h₁—enthalpy of steam in hydrogen reaction in kJ/kg;
h₂—enthalpy of industrial water at room temperature in kJ/kg;
C—specific heat capacity of flue gas in kJ/(kg·° C.);
Mₛₘₒₖₑ—amount of flue gas entering the exhaust gas waste heat boiler in kg/h;
t₁—average temperature of flue gas at an inlet of the exhaust gas waste heat boiler in ° C.;
tₒ—average temperature of flue gas at an outlet of the exhaust gas waste heat boiler in ° C.; and
α—heat loss coefficient of flue gas.

6. The comprehensive energy supply system based on a hydrogen energy storage system using residual flue gas of a gas engine according to claim 2, wherein the calculating the amount of natural gas raw material to be introduced according to the chemical reaction formula of the hydrogen production from natural gas, combined with the total carbon flow in the natural gas and the mass flow of steam generated by the exhaust gas waste heat boiler specifically performed in a formula as follows:
the total carbon flow in the natural gas is calculated in a formula as follows:
$$\sum {V}_{c a r b o n} = {γ}_{C H 4} + {γ}_{C 2 H 6} \times 2 + {γ}_{C 3 H 8} \times 3 + {γ}_{C 4 H 10} \times 4 ,$$
where ΣV_{carbon} is the total carbon flow in the natural gas, γ_{CH4} is a volume percentage of methane in the natural gas, γ_{C2H6} is a volume percentage of ethane, γ_{C3H8} is a volume percentage of propane, and γ_{C4H10} is a volume percentage of butane;
the determining the steam-to-carbon ratio in hydrogen production from natural gas and calculating the volume flow of natural gas according to the calculation formula of the steam-to-carbon ratio combined with the total carbon flow in the natural gas and a volume flow of steam is performed in a formula as follows:
$$ξ = \frac{{V}_{steam}}{{V}_{natural} \times \sum {V}_{carbon}} ,$$
where ξ is the steam-to-carbon ratio, which is empirically determined to be a constant, Vₙₐₜᵤᵣₐₗ is the volume flow of natural gas in Nm³/h, and Vₛₜₑₐₘ is the volume flow of steam in Nm³/h.

7. The comprehensive energy supply system based on a hydrogen energy storage system using residual flue gas of a gas engine according to claim 1, wherein the reforming hydrogen production device further includes a natural gas pipeline, a desulfurizer, a mixer for desulfurized natural gas and steam, a gas-liquid separator, a hydrogen purification apparatus, and a waste gas collector, wherein
a first input end of the mixer for desulfurized natural gas and steam is connected with a steam output end of the exhaust gas waste heat boiler, and an output end of the mixer for desulfurized natural gas and steam is connected with the hydrogenation reaction combustion furnace;
a first output end of the natural gas pipeline is connected to the desulfurizer, and an output end of the desulfurizer is connected to a second input end of the mixer for desulfurized natural gas and steam;
the hydrogenation reaction combustion furnace comprises a high-temperature reaction furnace and a flue gas channel, an input end of the high-temperature reaction furnace is connected with the output end of the mixer for desulfurized natural gas and steam, and a flue gas output end of the flue gas channel is connected with the exhaust gas waste heat boiler, an output end of the high-temperature reaction furnace is connected with an input end of the medium temperature shift converter, an output end of the medium temperature shift converter is connected with an input end of the gas-liquid separator, and an output end of the gas-liquid separator is connected with an input end of the hydrogen purification apparatus; and
a first output end of the hydrogen purification apparatus is connected with a hydrogen energy power generation apparatus, a second output end of the hydrogen purification apparatus is connected with the waste gas collector, and an output end of the waste gas collector is connected with the natural gas pipeline.

8. The comprehensive energy supply system based on a hydrogen energy storage system using residual flue gas of a gas engine according to claim 7, further comprising a hydrogen compressor and a natural gas compressor, wherein
an input end of the hydrogen compressor is connected with the hydrogen purification apparatus, and an output end of the hydrogen compressor is connected with the hydrogen energy power generation apparatus; and
an input end of the natural gas compressor is connected with the natural gas pipeline, and an output end of the natural gas compressor is connected with an input end of the desulfurizer.

9. The comprehensive energy supply system based on a hydrogen energy storage system using residual flue gas of a gas engine according to claim 7, further comprising a first heat exchanger, which is installed in a connecting pipeline between the high-temperature reaction furnace and the mixer for desulfurized natural gas and steam;
a second heat exchanger comprising a first cooling channel and a second cooling channel, wherein the first cooling channel is installed in a connecting pipeline between the desulfurizer and the high-temperature reaction furnace, and the second cooling channel is installed in a connecting pipeline between a compressor and the medium temperature shift converter; and
a third heat exchanger installed in a connecting pipeline between the gas-liquid separator and the medium temperature shift converter.

10. The comprehensive energy supply system based on a hydrogen energy storage system using residual flue gas of a gas engine according to claim 9, further comprising a thermal energy storage tank, wherein one end of the third heat exchanger is a cooling water input end, and the other end of the third heat exchanger is output to one of input ends of the thermal storage tank, and another of the input ends of the thermal storage tank is connected with a thermal energy output end of the hydrogen energy power generation apparatus.
